(19) United States
(12) Patent Application Publication
(10) Pub. No.: US 2012/0109235 A1
(43) Pub. Date: May 3, 2012

(54) CAPTURE DETECTION IN RESPONSE TO LEAD RELATED CONDITIONS

(75) Inventors: Todd J. Sheldon, North Oaks, MN (US); Bruce D. Gunderson, Plymouth, MN (US)

(73) Assignee: MEDTRONIC, INC., Minneapolis, MN (US)

(21) Appl. No.: 12/913,400

(22) Filed: Oct. 27, 2010

(51) Int. Cl. A61N 1/37; A61N 1/39

(52) U.S. Cl. 607/4; 607/28

(57) ABSTRACT

Various techniques for detecting cardiac capture in response to a detected lead related condition are described. One example method described includes delivering a pacing therapy to a heart of a patient, periodically determining whether the pacing therapy captures the heart of the patient, detecting a lead related condition, and, in response to the detected lead related condition, increasing a frequency of determining whether the pacing therapy captures the heart.

### TECHNICAL FIELD

This disclosure relates to medical devices and, more particularly, to medical devices that deliver therapeutic electrical signals to the heart.

### BACKGROUND

A variety of medical devices for delivering a therapy and/or monitoring a physiological condition have been used clinically or proposed for clinical use in patients. Examples include medical devices that deliver therapy to and/or monitor conditions associated with the heart, muscle, nerve, brain, stomach or other organs or tissues. Some therapies include the delivery of electrical signals, e.g., stimulation, to such organs or tissues. Some medical devices may employ one or more elongated electrical leads carrying electrodes for the delivery of therapeutic electrical signals to such organs or tissues, electrodes for sensing intrinsic electrical signals within the patient, which may be generated by such organs or tissue, and/or other sensors for sensing physiological parameters of a patient.

Medical leads may be configured to allow electrodes or other sensors to be positioned at desired locations for delivery of therapeutic electrical signals or sensing. For example, electrodes or sensors may be carried at a distal portion of a lead. A proximal portion of the lead may be coupled to a medical device housing, which may contain circuitry such as signal generation and/or sensing circuitry. In some cases, the medical leads and the medical device housing are implantable within the patient. Medical devices with a housing configured for implantation within the patient may be referred to as implantable medical devices.

Implantable cardiac pacemakers or cardioverter-defibrillators, for example, provide therapeutic electrical signals to the heart via electrodes carried by one or more implantable medical leads. The therapeutic electrical signals may include pulses or shocks for pacing, cardioversion, or defibrillation. In some cases, a medical device may sense intrinsic depolarizations of the heart, and control delivery of therapeutic signals to the heart based on the sensed depolarizations. Upon detection of an abnormal rhythm, such as bradycardia, tachycardia or fibrillation, an appropriate therapeutic electrical signal or signals may be delivered to restore or maintain a more normal rhythm. For example, in some cases, an implantable medical device may deliver pacing stimulation to the heart of the patient upon detecting tachycardia or bradycardia, and deliver cardioversion or defibrillation shocks to the heart upon detecting fibrillation.

Implantable cardiac pacemakers may also detect whether the pacing pulses have captured the cardiac tissue, i.e., resulted in depolarizations or contractions of the cardiac tissue. Various methods exist for detecting whether a pacing stimulus has captured the heart and determining capture thresholds. In some examples, a first pair of electrodes delivers a pacing pulse to a chamber, and the same or a different pair of electrodes detects an electrical signal, e.g., evoked response, in the chamber indicative of capture. In other examples, a device detects a mechanical contraction of the heart at the target site as evidence of capture of the heart by the pacing stimulus. In general, capture threshold determination or management involves delivery of pacing stimuli at incrementally increasing or decreasing magnitudes, e.g., voltage or current amplitudes or pulse widths, and identification of the magnitude at which capture or loss of capture occurs.

Implantable medical leads typically include a lead body containing one or more elongated electrical conductors that extend through the lead body from a connector assembly provided at a proximal lead end to one or more electrodes located at the distal lead end or elsewhere along the length of the lead body. The conductors connect signal generation and/or sensing circuitry within an associated implantable medical device housing to respective electrodes or sensors. Some electrodes may be used for both delivery of therapeutic signals and sensing. Each electrical conductor is typically electrically isolated from other electrical conductors and is encased within an outer sheath that electrically insulates the lead conductors from body tissue and fluids.

Medical lead bodies implanted for cardiac applications tend to be continuously flexed by the beating of the heart. Other stresses may be applied to the lead body, including the conductors therein, during implantation or lead repositioning. Patient movement can cause the route traversed by the lead body to be constricted or otherwise altered, causing stresses on the lead body and conductors. In rare instances, such stresses may fracture a conductor within the lead body. The fracture may be continuously present, or may intermittently manifest as the lead flexes and moves. Also, the wear and degradation of the insulation between the conductors may result in shorting.

Additionally, the electrical connection between medical device connector elements and the lead connector elements can be intermittently or continuously disrupted. For example, connection mechanisms, such as set screws, may be insufficiently tightened at the time of implantation, followed by a gradual loosening of the connection. Also, lead pins may not be completely inserted.

Lead fracture, disrupted connections, or other causes of short circuits or open circuits may be referred to, in general, as lead related conditions. In the case of cardiac leads, sensing of an intrinsic heart rhythm through a lead can be altered by lead related conditions. Identifying lead related conditions may be challenging, particularly in a clinic, hospital or operating room setting, due to the often intermittent nature of lead related conditions. Identification of lead related conditions may allow modifications of the therapy or sensing, or lead replacement.

### SUMMARY

In general, the disclosure is directed toward modifying the evaluation of cardiac capture in response to detection of a lead related condition. For example, a frequency of capture detection may be increased in response to detection of a lead related condition.

In one example, a method comprises delivering a pacing therapy to a heart of a patient, periodically determining whether the pacing therapy captures the heart of the patient, detecting a lead related condition, and, in response to the detection of the lead related condition, increasing a frequency of determining whether the pacing therapy captures the heart.

In another example, a system comprises a stimulation generator configured to deliver a pacing therapy to a heart of a patient, an electrical stimulation lead coupled to the stimulation generator, wherein the stimulation generator delivers the pacing signal via the electrical stimulation lead, a sensing module configured to sense an indicator of integrity of the lead, and a processor configured to periodically determine whether the pacing therapy captures the heart of the patient, identify a lead related condition based on the sensed indicator, and increase a frequency of determining whether the pacing therapy captures the heart in response to the detection of the lead related condition.

In another example, a system comprises means for delivering a pacing therapy to a heart of a patient, means for periodically determining whether the pacing therapy captures the heart of the patient, means for detecting a lead related condition, and means for increasing a frequency of determining whether the pacing therapy captures the heart in response to the detection of the lead related condition.

In another example, a computer-readable storage medium contains instructions. When executed, the instructions cause a programmable processor to deliver a pacing therapy to a heart of a patient, periodically determine whether the pacing therapy captures the heart of the patient, detect a lead related condition, and, in response to the detection of the lead related condition, increase a frequency of determining whether the pacing therapy captures the heart.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual drawing illustrating an example system that includes an implantable medical device (IMD) coupled to implantable medical leads.

FIG. 2 is a conceptual drawing illustrating the example IMD and leads of FIG. 1 in conjunction with a heart.

FIG. 3 is a conceptual drawing illustrating the example IMD of FIG. 1 coupled to a different example configuration of two implantable medical leads in conjunction with a heart.

FIG. 4 is a functional block diagram illustrating an example configuration of the IMD of FIG. 1.

FIG. 5 is a functional block diagram illustrating an example configuration of an external programmer that facilitates user communication with the IMD.

FIG. 6 is a block diagram illustrating an example system that includes an external device, such as a server, and one or more computing devices that are coupled to the IMD and programmer shown in FIG. 1 via a network.

FIG. 7 is a flow diagram of an example method of increasing a frequency of capture detection in response to a lead related condition.

### DETAILED DESCRIPTION

Implantable cardiac pacemakers and other cardiac devices may be capable of substantially continuous monitoring of cardiac capture. For example, a cardiac device may be capable of sensing the timing and amplitude of an evoked response on a beat-beat basis. However, since loss of capture is rare under normal conditions, the device may be programmed to detect capture on a less frequent basis, e.g., daily.

A lead related condition may result in an increased pacing threshold and/or loss of capture. Therefore, in response to detection of a lead related condition, a frequency of capture detection may be increased, e.g., from a first frequency of periodic capture detection to a second, greater frequency of capture detection. In some examples, capture is detected on a substantially continuous, e.g., on an every beat basis, in response to detection of a lead related condition.

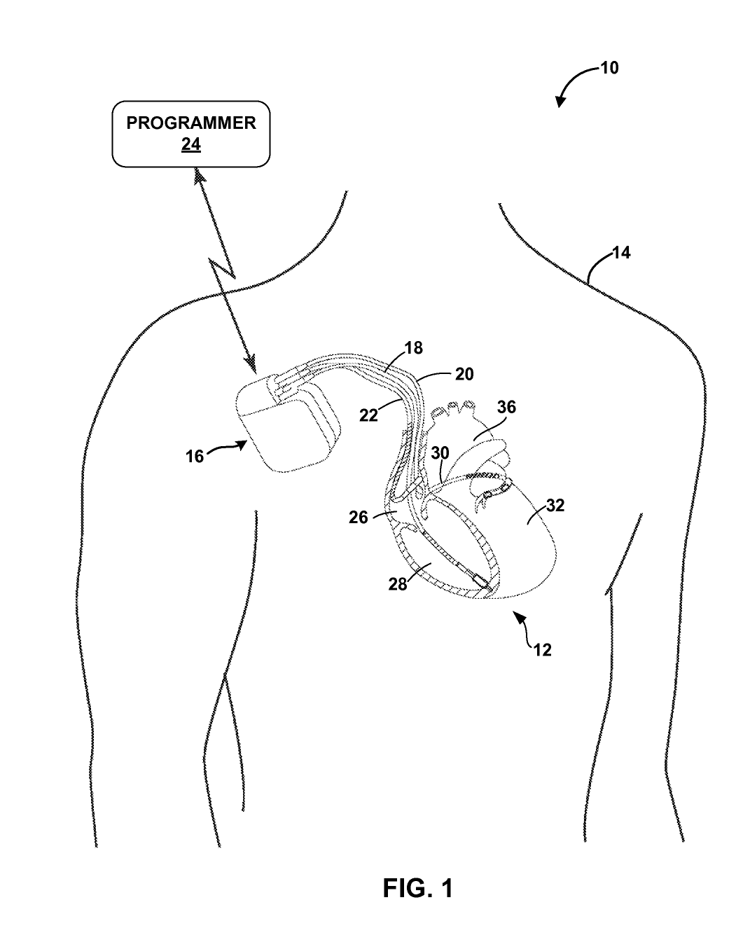

FIG. 1 is a conceptual diagram illustrating an example system 10 that may be used for sensing electrogram (EGM) signals of a patient 14 and/or to provide therapy to a heart 12 of patient 14. System 10 includes an IMD 16, which is coupled to leads 18, 20, and 22, as well as to a programmer 24. IMD 16 may be, for example, an implantable pacemaker, a cardioverter, and/or a defibrillator that provides electrical signals to heart 12 via electrodes coupled to one or more of leads 18, 20, and 22. Patient 14 is ordinarily, but not necessarily, a human patient.

Leads 18, 20, 22 extend into heart 12 to sense electrical activity of heart 12 and/or deliver electrical stimulation to heart 12. In the example shown in FIG. 1, right ventricular (RV) lead 18 extends through one or more veins (not shown), the superior vena cava (not shown), and right atrium 26, and into right ventricle 28. Left ventricular (LV) coronary sinus lead 20 extends through one or more veins, the vena cava, right atrium 26, and into coronary sinus 30 to a region adjacent to the free wall of left ventricle 32 of heart 12. Right atrial (RA) lead 22 extends through one or more veins and the vena cava, and into right atrium 26 of heart 12.

In some examples, system 10 may additionally or alternatively include one or more leads or lead segments (not shown in FIG. 1) that deploy one or more electrodes within the vena cava or other vein. These electrodes may allow alternative electrical sensing configurations that may provide improved or supplemental sensing in some patients. Furthermore, in some examples, therapy system 10 may include temporary or permanent epicardial or subcutaneous leads, instead of or in addition to leads 18, 20 and 22. Such leads may be used for one or more of cardiac sensing, pacing, or cardioversion/defibrillation.

IMD 16 may sense electrical signals attendant to the depolarization and repolarization of heart 12 via electrodes (not shown in FIG. 1) coupled to at least one of leads 18, 20, 22. In some examples, IMD 16 provides pacing pulses to heart 12 based on the electrical signals sensed within heart 12. IMD 16 may sense electrical signals attendant to the depolarization and repolarization of heart 12 subsequent to pacing pulses to determine whether the pacing pulses captured the cardiac tissue, e.g., caused a depolarization referred to as an evoked response. The configurations of electrodes used by IMD 16 for sensing and pacing may be unipolar or bipolar. IMD 16 may detect arrhythmia of heart 12, such as tachycardia or fibrillation of ventricles 28 and 32, and may also provide defibrillation therapy and/or cardioversion therapy via electrodes located on at least one of leads 18, 20, 22. In some examples, IMD 16 may be programmed to deliver a progression of therapies, e.g., pulses with increasing energy levels, until a fibrillation of heart 12 is stopped. IMD 16 detects fibrillation employing one or more fibrillation detection techniques known in the art.

IMD 16 may also sense indicators of lead related conditions. For example, IMD 16 may perform one or more impedance measurements for one or more of leads 18, 20, 22 as a lead integrity test. In some examples, IMD 16 may compare a measured impedance to a threshold to determine whether lead(s) 18, 20, 22 have a lead related condition. If the impedance measurements indicate a lead related condition, IMD 16 may increase a frequency of capture detection as described in this disclosure. IMD 16 may also provide an alert, change a sensing configuration, e.g., a vector used for sensing, change a therapy configuration, e.g., a vector used for delivery of a therapeutic signal, or withhold any responsive therapeutic shocks to the patient in response to detecting a lead related condition. As another example, IMD 16 may change a mode of therapy delivery to a mode that would not inhibited by oversensing of ventricular events caused by a lead related condition of one of ventricular leads 18 and 20, such as VVO pacing, VVT pacing, or ventricular sense response in cardiac resynchronization devices.

In some examples, IMD 16 monitors other parameters, such as the number or frequency non-sustained high-rate cardiac episodes or short cardiac intervals to detect lead related conditions. Short cardiac intervals may include R-R intervals below a threshold, e.g., such that they are considered to be non-physiologic. A non-sustained high-rate cardiac episodes may comprise a series of R-R intervals below a tachycardia or fibrillation threshold, where the series was not sustained for enough intervals to result in detection of a tachycardia or fibrillation.

Programmer 24 can be a handheld computing device, a computer workstation, or a networked computing device. Programmer 24 can include a user interface that receives input from a user, which can include a keypad and a suitable display such as, for example, a touch screen display. Programmer 24 can additionally or alternatively include a peripheral pointing device, such as a mouse, via which a user may interact with the user interface. The user may also interact with programmer 24 remotely via a networked computing device.

A user, such as a physician, technician, surgeon, electrophysiologist, or other clinician, may interact with programmer 24 to communicate with IMD 16. For example, the user may interact with programmer 24 to retrieve physiological or diagnostic information from IMD 16. A user may also interact with programmer 24 to program IMD 16, e.g., select values for operational parameters of the IMD.

For example, the user may use programmer 24 to retrieve information from IMD 16 regarding the rhythm of heart 12, trends therein over time, or arrhythmic episodes. As another example, the user may use programmer 24 to retrieve information from IMD 16 regarding other sensed physiological parameters of patient 14, such as intracardiac or intravascular pressure, activity, posture, respiration, or thoracic impedance. As another example, the user may use programmer 24 to retrieve information from IMD 16 regarding the performance or integrity of IMD 16 or other components of system 10, such as leads 18, 20 and 22, or a power source of IMD 16. In some examples, this information may be presented to the user as an alert. For example, a lead-related condition indicated by a lead integrity test by IMD 16 may cause programmer 24 to provide an alert to a user.

As another example, the user may use programmer 24 to retrieve information from IMD 16 regarding cardiac capture, such as times that loss of capture occurred, frequency of loss of capture, and pacing threshold values for alternate pacing electrode configurations. In some examples, this information may include indications of whether a capture threshold increased and/or capture was lost subsequent to detection of a lead related condition.

The user may use programmer 24 to program a therapy progression, select electrodes used to deliver defibrillation pulses, select waveforms for the defibrillation pulse, or select or configure a fibrillation detection algorithm for IMD 16. The user may also use programmer 24 to program aspects of other therapies provided by IMD 16, such as cardioversion or pacing therapies. In some examples, the user may activate certain features of IMD 16 by entering a single command via programmer 24, such as depression of a single key or combination of keys of a keypad or a single point-and-select action with a pointing device.

IMD 16 and programmer 24 may communicate via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the implant site of IMD 16 in order to improve the quality or security of communication between IMD 16 and programmer 24.

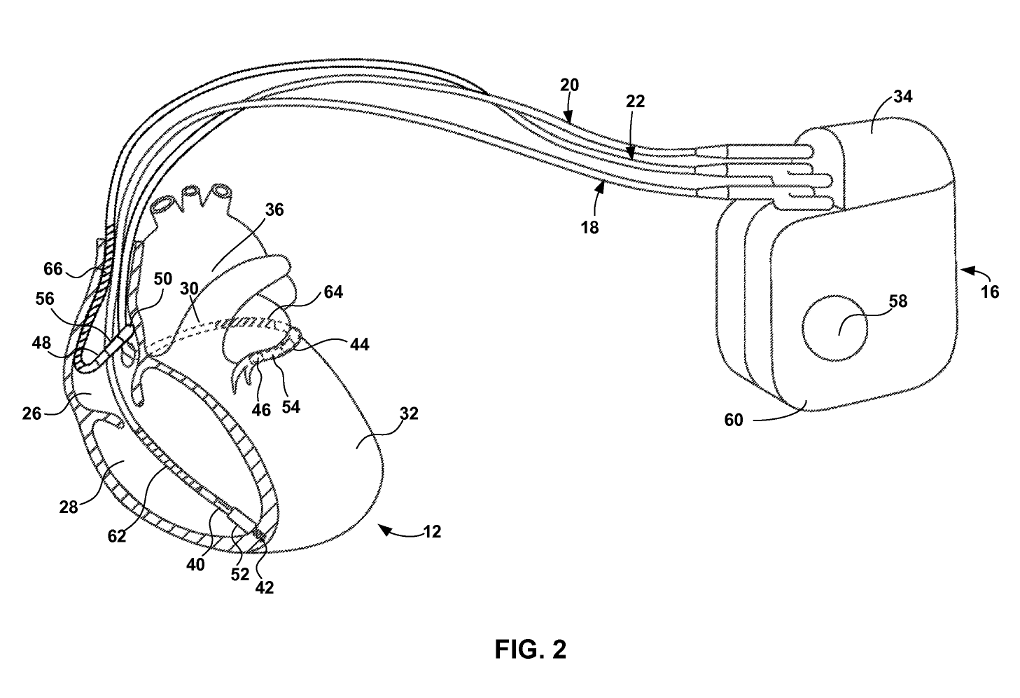

FIG. 2 is a conceptual diagram illustrating IMD 16 and leads 18, 20, 22 of therapy system 10 in greater detail. Leads 18, 20, 22 may be electrically coupled to a stimulation generator, a sensing module, or other modules of IMD 16 via a connector block 34. The proximal ends of leads 18, 20, 22 may include electrical contacts that electrically couple to respective electrical contacts within connector block 34. In addition, leads 18, 20, 22 may be mechanically coupled to connector block 34 with set screws, connection pins or another suitable mechanical coupling mechanism.

Each of leads 18, 20, 22 includes an elongated insulative lead body, which may carry a number of concentric coiled conductors separated from one another by tubular insulative sheaths. Bipolar electrodes 40 and 42 are located proximate to a distal end of lead 18. In addition, bipolar electrodes 44 and 46 are located proximate to a distal end of lead 20 and bipolar electrodes 48 and 50 are located proximate to a distal end of lead 22.

Electrodes 40, 44 and 48 may be ring electrodes, and electrodes 42, 46 and 50 may be extendable helix tip electrodes mounted retractably within insulative electrode heads 52, 54 and 56, respectively. Each of electrodes 40, 42, 44, 46, 48 and 50 may be electrically coupled to a respective one of the coiled conductors within the lead body of its associated lead 18, 20, 22, and thereby coupled to respective ones of the electrical contacts on the proximal end of leads 18, 20 and 22.

Electrodes 40, 42, 44, 46, 48 and 50 may sense electrical signals attendant to the depolarization and repolarization of heart 12. The electrical signals are conducted to IMD 16 via the respective leads 18, 20, 22. In some examples, IMD 16 also delivers pacing pulses via electrodes 40, 42, 44, 46, 48 and 50 to cause depolarization of cardiac tissue of heart 12. Electrodes 40, 42, 44, 46, 48 and 50 may sense evoked responses subsequent to pacing pulses to detect whether the pacing pulses captured the tissue of heart 12, e.g., causing a depolarization.

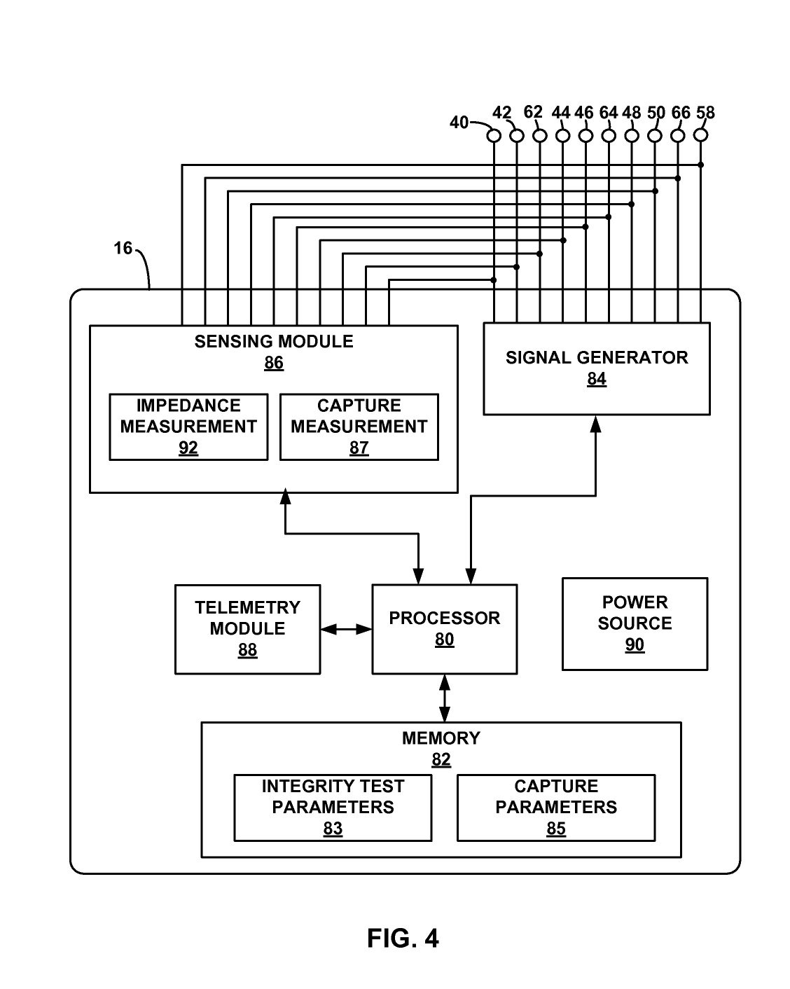

In some examples, as illustrated in FIG. 2, IMD 16 includes one or more housing electrodes, such as housing electrode 58, which may be formed integrally with an outer surface of a hermetically-sealed housing 60 of IMD 16 or otherwise coupled to housing 60. In some examples, housing electrode 58 is defined by an uninsulated portion of an outward facing portion of housing 60 of IMD 16. Other divisions between insulated and uninsulated portions of housing 60 may be employed to define two or more housing electrodes. In some examples, housing electrode 58 can include substantially all of housing 60. Any of electrodes 40, 42, 44, 46, 48 and 50 may be used for unipolar sensing or pacing in combination with housing electrode 58. As described in further detail with reference to FIG. 4, housing 60 may enclose a stimulation generator that generates cardiac pacing pulses and defibrillation or cardioversion shocks, as well as a sensing module for monitoring the patient's heart rhythm.

Leads 18, 20, 22 also include elongated electrodes 62, 64, 66, respectively, which may be a coil. IMD 16 may deliver defibrillation pulses to heart 12 via any combination of elongated electrodes 62, 64, 66, and housing electrode 58. Electrodes 58, 62, 64, 66 may also be used to deliver cardioversion pulses to heart 12. Electrodes 62, 64, 66 may be fabricated from any suitable electrically conductive material, such as, but not limited to, platinum, platinum alloy or other materials known to be usable in implantable defibrillation electrodes. In some examples, electrodes 62, 64 and 66 may be used for pacing or sensing in combination with any of electrodes 40, 42, 44, 46, 48, 50 and 58.

IMD 16 may also sense indicators of lead related conditions. For example, IMD 16 may measure the impedance of one or more electrical paths, each path including two or more of electrodes 40, 42, 44, 46, 48, 50, 58, 62, 64, and 66 as a lead integrity test. In some examples, IMD 16 may compare a measured impedance to a threshold to determine whether lead(s) 18, 20, 22 have a lead related condition. If the impedance measurements indicate a lead related condition, IMD 16 may increase a frequency of capture detection as described in this disclosure. IMD 16 may also provide an alert, change a sensing configuration, change a therapy configuration, or withhold any responsive therapeutic shocks to the patient in response to detecting a lead related condition. As another example, IMD 16 may change a mode of therapy delivery to a mode that would not inhibited by oversensing of ventricular events caused by a lead related condition of one of ventricular leads 18 and 20, such as, e.g., VVO pacing, VVT pacing, or ventricular sense response in cardiac resynchronization devices. In some examples, IMD 16 monitors other parameters, such as the number or frequency non-sustained high-rate cardiac episodes or short cardiac intervals to detect lead related conditions.

The configuration of therapy system 10 illustrated in FIGS. 1 and 2 is merely one example. In other examples, a therapy system may include epicardial leads and/or patch electrodes instead of or in addition to transvenous leads 18, 20, 22 illustrated in FIG. 1. Further, IMD 16 need not be implanted within patient 14. In examples in which IMD 16 is not implanted in patient 14, IMD 16 may deliver defibrillation pulses and other therapies to heart 12 via percutaneous leads that extend through the skin of patient 14 to a variety of positions within or outside of heart 12.

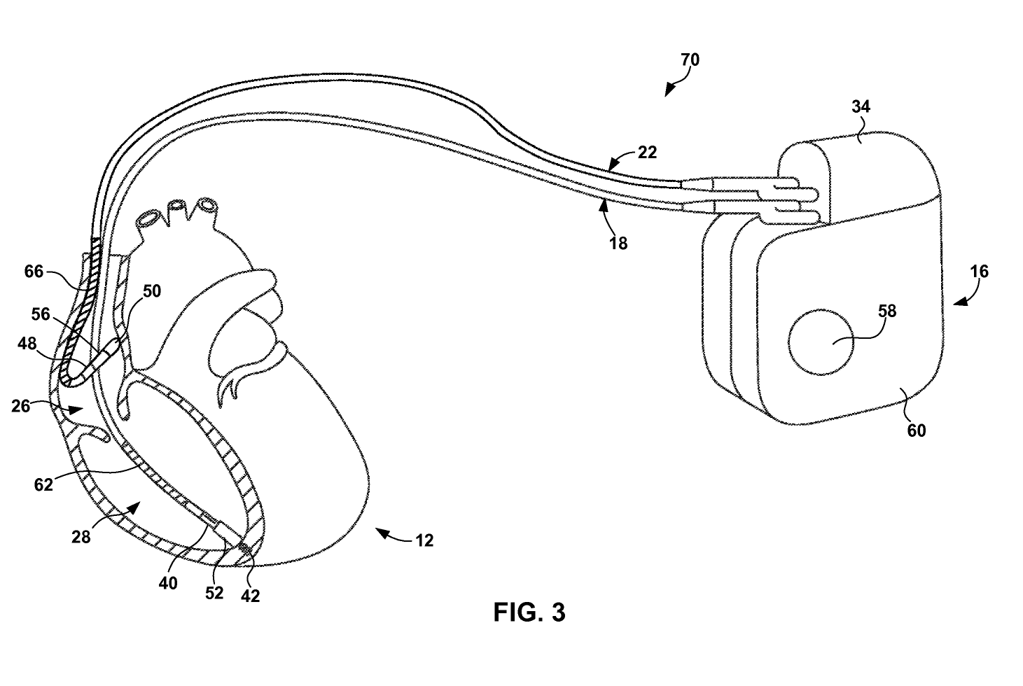

In other examples of therapy systems that provide electrical stimulation therapy to heart 12, a therapy system may include any suitable number of leads coupled to IMD 16, and each of the leads may extend to any location within or proximate to heart 12. For example, other examples of therapy systems may include three transvenous leads located as illustrated in FIGS. 1 and 2, and an additional lead located within or proximate to left atrium 33. In other examples, a therapy system may include epicardial leads and/or patch electrodes instead of or in addition to transvenous leads 18, 20, 22 illustrated in FIG. 1. As another example, other examples of therapy systems may include a single lead that extends from IMD 16 into right atrium 26 or right ventricle 28, or two leads that extend into a respective one of right ventricle 26 and right atrium 28. An example of this type of therapy system is shown in FIG. 3.

FIG. 3 is a conceptual diagram illustrating another example of a therapy system 70, which is similar to therapy system 10 of FIGS. 1-2, but includes two leads 18, 22, rather than three leads. Leads 18, 22 are implanted within right ventricle 28 and right atrium 26, respectively. Therapy system 70 shown in FIG. 3 may be useful for providing defibrillation and pacing pulses to heart 12.

Further, in some examples, IMD 16 need not be coupled to endocardial or epicardial leads, and may instead be coupled to leads that carry one or more electrodes and are implanted subcutaneously without having to surgically invade the thoracic cavity or vasculature. In such subcutaneously implanted apparatuses, IMD 16 may deliver defibrillation pulses, pacing, and other therapies to heart 12 via the subcutaneous leads.

FIG. 4 is a functional block diagram illustrating an example configuration of IMD 16. In the illustrated example, IMD 16 includes processor 80, memory 82, signal generator 84, sensing module 86, telemetry module 88, and power source 90. Memory 82 includes computer-readable instructions that, when executed by processor 80, cause IMD 16 and processor 80 to perform various functions described herein. Memory 82 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media.

Processor 80 may include any one or more of a microprocessor, a controller, a digital signal processor (DSP), an application specific integrated circuit (ASIC), a field-programmable gate array (FPGA), or equivalent discrete or analog logic circuitry. In some examples, processor 80 may include multiple components, such as any combination of one or more microprocessors, one or more controllers, one or more DSPs, one or more ASICs, or one or more FPGAs, as well as other discrete or integrated logic circuitry. The functions attributed to processor 80 herein may be embodied as software, firmware, hardware or any combination thereof.

Processor 80 controls signal generator 84 to deliver stimulation therapy to heart 12 according to a selected one or more therapy programs, which may be stored in memory 82. For example, processor 80 may control signal generator 84 to deliver electrical pulses with the amplitudes, pulse widths, frequency, or electrode polarities specified by the selected one or more therapy programs.

Signal generator 84 is electrically coupled to electrodes 40, 42, 44, 46, 48, 50, 58, 62, 64, and 66, e.g., via conductors of the respective leads 18, 20, 22, or, in the case of housing electrode 58, via an electrical conductor disposed within housing 60 of IMD 16. Signal generator 84 generates and delivers electrical stimulation therapy to heart 12. For example, signal generator 84 may deliver defibrillation shocks as therapy to heart 12 via at least two electrodes 58, 62, 64, 66. Signal generator 84 may deliver pacing pulses via ring electrodes 40, 44, 48 coupled to leads 18, 20, and 22, respectively, and/or helical electrodes 42, 46, and 50 of leads 18, 20, and 22, respectively. In some examples, signal generator 84 delivers pacing, cardioversion, or defibrillation stimulation in the form of electrical pulses. In other examples, signal generator 84 may deliver one or more of these types of stimulation in the form of other signals, such as sine waves, square waves, or other substantially continuous time signals.

Signal generator 84 may include a switch module, and processor 80 may use the switch module to select, e.g., via a data/address bus, which of the available electrodes are used to deliver defibrillation pulses or pacing pulses. The switch module may include a switch array, switch matrix, multiplexer, or any other type of switching device suitable to selectively couple stimulation energy to selected electrodes.

Electrical sensing module 86 monitors signals from at least one of electrodes 40, 42, 44, 46, 48, 50, 58, 62, 64 or 66 to monitor electrical activity of heart 12. Sensing module 86 may also include a switch module to select which of the available electrodes are used to sense the heart activity, depending upon which electrode combination is used in the current sensing configuration. In some examples, processor 80 may select the electrodes that function as sense electrodes, i.e., select the sensing configuration, via the switch module within sensing module 86. Processor 80 may control the functionality of sensing module 86 by providing signals via a data/address bus.

Sensing module 86 may include one or more detection channels, each of which may include an amplifier. The detection channels may be used to sense the cardiac signals. Some detection channels may detect cardiac events, such as R- or P-waves, and provide indications of the occurrences of such events to processor 80. One or more other detection channels may provide the signals to an analog-to-digital converter, for processing or analysis by processor 80. In response to the signals from processor 80, the switch module within sensing module 86 may couple selected electrodes to selected detection channels.

For example, sensing module 86 may include one or more narrow band channels, each of which may include a narrow band filtered sense-amplifier that compares the detected signal to a threshold. If the filtered and amplified signal is greater than the threshold, the narrow band channel indicates that a certain electrical cardiac event, e.g., depolarization, has occurred. Processor 80 then uses that detection in measuring frequencies of the sensed events. Different narrow band channels of sensing module 86 may have distinct functions. For example, some various narrow band channels may be used to sense either atrial or ventricular events.

In one example, at least one narrow band channel may include an R-wave amplifier that receives signals from the sensing configuration of electrodes 40 and 42, which are used for sensing and/or pacing in right ventricle 28 of heart 12. Another narrow band channel may include another R-wave amplifier that receives signals from the sensing configuration of electrodes 44 and 46, which are used for sensing and/or pacing proximate to left ventricle 32 of heart 12. In some examples, the R-wave amplifiers may take the form of an automatic gain controlled amplifier that provides an adjustable sensing threshold as a function of the measured R-wave amplitude of the heart rhythm.

In addition, in some examples, a narrow band channel may include a P-wave amplifier that receives signals from electrodes 48 and 50, which are used for pacing and sensing in right atrium 26 of heart 12. In some examples, the P-wave amplifier may be an automatic gain controlled amplifier that provides an adjustable sensing threshold as a function of the measured P-wave amplitude of the heart rhythm. Examples of R-wave and P-wave amplifiers are described in U.S. Pat. No. 5,117,824 to Keimel et al., which issued on Jun. 2, 1992 and is entitled, “APPARATUS FOR MONITORING ELECTRICAL PHYSIOLOGIC SIGNALS,” and is incorporated herein by reference in its entirety. Other amplifiers may also be used.

One or more of the sensing channels of sensing module 86 may also be selectively coupled to housing electrode 58, or elongated electrodes 62, 64, or 66, with or instead of one or more of electrodes 40, 42, 44, 46, 48 or 50, e.g., for unipolar sensing of R-waves or P-waves in any of chambers 26, 28, or 32 of heart 12.

One or more sensing channels of sensing module 86, e.g., one or more narrow band channels, may sense evoked responses to detect capture and/or inadequate capture when signal generator 84 delivers a pacing pulse. For example, processor 80 may control which of electrodes 40, 42, 44, 46, 48, 50, 58, 62, 64, and 66 is coupled to electrical sensing module 86 to detect an evoked electrical response to a pacing pulse. Memory 82 may store capture parameters 85, such as predetermined intervals or voltage thresholds which define whether a detected signal has an adequate magnitude and is appropriately timed relative to the pacing pulse to be considered an evoked response. In some examples, a channel of electrical sensing module 86 used to detect capture comprises an amplifier which provides an indication to processor 80 when a detected signal has an adequate magnitude within an appropriately timed interval relative to the pacing pulse.

Processor 80 controls the selection of electrode configurations for delivering pacing pulses and for detecting capture and/or loss of capture (LOC). Processor 80, for example, may communicate with signal generator 84 to select two or more stimulation electrodes in order to generate one or more pacing pulses for delivery to a selected chamber of heart 12. Processor 80 may also communicate with electrical sensing module 86 to select two or more sensing electrodes for capture detection based on the chamber to which the pacing pulse is delivered by signal generator 84.

In the example of FIG. 4, sensing module 86, and in particular one or more capture measurement channels 87 of sensing module 86, is capable of detecting capture and LOC. For example, one or more capture measurement channels 87 may detect the amplitude and timing of an evoked response. Memory 82 may store capture parameters 85, such as predetermined intervals or voltage thresholds which define whether a detected signal has an adequate magnitude and is appropriately timed relative to the pacing pulse to be considered an evoked response. In some examples, one or more capture measurement channels 87 of electrical sensing module 86 comprises an amplifier which provides an indication to processor 80 when a detected signal has an adequate magnitude within an appropriately timed interval relative to the pacing pulse.

Processor 80 may control a frequency of capture detection. Under normal conditions, processor 80 and one or more capture measurement channels 87 may detect cardiac capture on a periodic basis, e.g., at a first frequency. For example, processor 80 and one or more capture measurement channels 87 may verify that a selected pacing parameter set is capturing the heart on approximately a daily basis. In response to detection of a lead related condition, processor 80 may increase the frequency of capture detection to a second, higher frequency. For example, processor 80 and one or more capture measurement channels 87 may detect cardiac capture on a substantially continuous basis, e.g., on approximately an every heartbeat basis, subsequent to detection of a lead related condition. In other examples, processor 80 and one or more capture measurement channels 87 may detect capture on a periodic basis with an increased frequency, e.g., approximately every fifteen minutes, or approximately every one minute, subsequent to detection of a lead related condition. Since increased pacing capture thresholds and/or loss of capture may occur subsequent to a lead related condition, increasing a frequency of capture detection in response to detecting a lead related condition may allow processor 80 to detect and respond to these issues.

Processor 80 may also control signal generator 84 to deliver pacing pulses at various magnitudes, e.g., voltage amplitudes, to determine a pacing capture threshold. This process of determining a pacing capture threshold may be referred to as a pacing threshold search. One or more capture measurement channels 87 may detect whether capture and/or LOC occurs. According to some examples, if an initial pacing pulse captured, then processor 80 may incrementally decrease the magnitude, e.g., voltage amplitude, of the pacing pulse until LOC is detected. If the initial pacing pulse did not capture, then processor 80 may incrementally increase the magnitude until capture occurs. Any known techniques for performing a pacing threshold search may be utilized with the techniques of this disclosure.

Processor 80 may determine a voltage at which capture/LOC occurs, which may be referred to as the pacing capture threshold. This pacing threshold search process may allow processor 80 and one or more capture measurement channels 87 of sensing module 86 to quickly and accurately determine the estimated tissue pacing capture thresholds for one or more pacing vector configurations, thereby allowing a clinician to select particular electrode configuration for IMD 16 that will deliver sufficient energy to pace heart 12 without unnecessarily depleting power source 90. Although described herein primarily with reference to examples in which voltage amplitude is adjusted during the capture pacing threshold search to identify a voltage amplitude at which capture/LOC occurs, the techniques are applicable to examples in which any one or more parameters that effects the magnitude of the pacing stimulus, e.g., current amplitude or pulse width, are adjusted.

In some examples, processor 80 and one or more capture measurement channels 87 of sensing module 86 perform a capture pacing threshold search subsequent to a detected a lead related condition, e.g., to determine whether the capture threshold has increased due to the lead related condition.

In some examples, processor 80 may monitor trends in capture threshold values. For example, an increase in capture threshold, e.g., by a threshold amount or a threshold amount over a defined time period, may be indicative of a lead related condition. In some examples, processor 80 may identify lead related conditions based on capture threshold values, e.g., in combination with impedance measurements or other parameters.

In some examples, sensing module 86 includes a wide band channel, which may include an amplifier with a relatively wider pass band than the R-wave or P-wave amplifiers. Signals from the selected sensing electrodes that are selected for coupling to this wide-band amplifier may be converted to multi-bit digital signals by an analog-to-digital converter provided by, for example, sensing module 86 or processor 80. Processor 80 may store signals the digitized versions of signals from the wide band channel in memory 82 as EGM signals. The storage of such EGMs in memory 82 may be under the control of a direct memory access circuit.

In some examples, processor 80 may employ digital signal analysis techniques to characterize the digitized signals from the wide band channel to, for example, detect and classify the patient's heart rhythm. Processor 80 may detect and classify the patient's heart rhythm by employing any of the numerous signal processing methodologies known in the art.

If IMD 16 generates and delivers pacing pulses to heart 12, processor 80 may include pacer timing and control module, which may be embodied as hardware, firmware, software, or any combination thereof. The pacer timing and control module may comprise a dedicated hardware circuit, such as an ASIC, separate from other processor 80 components, such as a microprocessor, or a software module executed by a component of processor 80, which may be a microprocessor or ASIC. The pacer timing and control module may include programmable counters that control the basic time intervals associated with DDD, VVI, DVI, VDD, AAI, DDI, DDDR, VVIR, DVIR, VDDR, AAIR, DDIR and other modes of single and dual chamber pacing. In the aforementioned pacing modes, “D” may indicate dual chamber, “V” may indicate a ventricle, “I” may indicate inhibited pacing (e.g., no pacing), and “A” may indicate an atrium. The first letter in the pacing mode may indicate the chamber that is paced, the second letter may indicate the chamber that is sensed, and the third letter may indicate the chamber in which the response to sensing is provided.

Intervals defined by the pacer timing and control module within processor 80 may include atrial and ventricular pacing escape intervals, refractory periods during which sensed P-waves and R-waves are ineffective to restart timing of the escape intervals, and the pulse widths of the pacing pulses. As another example, the pace timing and control module may define a blanking period, and provide signals to sensing module 86 to blank one or more channels, e.g., amplifiers, for a period during and after delivery of electrical stimulation to heart 12. The durations of these intervals may be determined by processor 80 in response to stored data in memory 82. The pacer timing and control module of processor 80 may also determine the amplitude of the cardiac pacing pulses.

During pacing, escape interval counters within the pacer timing/control module of processor 80 may be reset upon sensing of R-waves and P-waves with detection channels of sensing module 86. Signal generator 84 may include pacer output circuits that are coupled, e.g., selectively by a switching module, to any combination of electrodes 40, 42, 44, 46, 48, 50, 58, 62, or 66 appropriate for delivery of a bipolar or unipolar pacing pulse to one of the chambers of heart 12. Processor 80 may reset the escape interval counters upon the generation of pacing pulses by signal generator 84, and thereby control the basic timing of cardiac pacing functions, including anti-tachyarrhythmia pacing.

The value of the count present in the escape interval counters when reset by sensed R-waves and P-waves may be used by processor 80 to measure the durations of R-R intervals, P-P intervals, PR intervals and R-P intervals, and these measurements may be stored in memory 82. Processor 80 may use the count in the interval counters to detect a suspected tachyarrhythmia event, such as ventricular fibrillation or ventricular tachycardia. A portion of memory 82 may be configured as a plurality of recirculating buffers, capable of holding series of measured intervals, which may be analyzed by processor 80 in response to the occurrence of a pace or sense interrupt to determine whether the patient's heart 12 is presently exhibiting atrial or ventricular tachyarrhythmia, or the like.

In some examples, an arrhythmia detection method may include any suitable tachyarrhythmia detection algorithms. In one example, processor 80 may utilize all or a subset of the rule-based detection methods described in U.S. Pat. No. 5,545,186 to Olson et al., entitled, “PRIORITIZED RULE BASED METHOD AND APPARATUS FOR DIAGNOSIS AND TREATMENT OF ARRHYTHMIAS,” which issued on Aug. 13, 1996, or in U.S. Pat. No. 5,755,736 to Gillberg et al., entitled, “PRIORITIZED RULE BASED METHOD AND APPARATUS FOR DIAGNOSIS AND TREATMENT OF ARRHYTHMIAS,” which issued on May 26, 1998. U.S. Pat. No. 5,545,186 to Olson et al. U.S. Pat. No. 5,755,736 to Gillberg et al. is incorporated herein by reference in their entireties. However, other arrhythmia detection methodologies may also be employed by processor 80.

Processor 80 may determine that tachyarrhythmia has occurred by identification of shortened R-R (or P-P) interval lengths. For example, processor 80 may detect tachycardia when the interval length falls below 320 milliseconds (ms) and fibrillation when the interval length falls below 280 ms. These interval lengths are merely examples, and a user may define the interval lengths as desired, which may then be stored within memory 82. This interval length may need to be detected for a certain number of consecutive cycles, for a certain percentage of cycles within a running window, or a running average for a certain number of cardiac cycles, as examples.

In the event that processor 80 detects an atrial or ventricular tachyarrhythmia based on signals from sensing module 86, and an anti-tachyarrhythmia pacing regimen is desired, timing intervals for controlling the generation of anti-tachyarrhythmia pacing therapies by signal generator 84 may be loaded by processor 80 into the pacer timing and control module to control the operation of the escape interval counters therein and to define refractory periods during which detection of R-waves and P-waves is ineffective to restart the escape interval counters. If IMD 16 generates and delivers cardioversion or defibrillation pulses to heart 12, signal generator 84 may include a high voltage charge circuit and a high voltage output circuit.

Processor 80 may monitor one or more indicators of lead integrity, such as lead impedance, the frequency of non-sustained high-rate cardiac episodes, the frequency of short intervals counted on a sensing integrity counter, open circuits, short circuits and/or capacitor droop. Although lead impedance is primarily described herein for purposes of example, any indicator of lead integrity may be monitored to detected lead related conditions.

Sensing module 86 and/or processor 80 are capable of collecting, measuring, and/or calculating impedance data for any of a variety of electrical paths that include two or more of electrodes 40, 42, 44, 46, 48, 50, 58, 62, 64 and 66. Impedance measurement module 92 can measure electrical parameter values during delivery of an electrical signal between at least two of the electrodes. Processor 80 may control signal generator 84 to deliver the electrical signal between the electrodes. Processor 80 may determine impedance values based on parameter values measured by impedance measurement module 92, and store the measured impedance values in memory 82.

In some examples, processor 80 may perform an impedance measurement by controlling delivery, from signal generator 84, of a voltage pulse between first and second electrodes. Impedance measurement module 92 may measure a resulting current, and processor 80 may calculate a resistance based upon the voltage amplitude of the pulse and the measured amplitude of the resulting current. In other examples, processor 80 may perform an impedance measurement by controlling delivery, from signal generator 84, of a current pulse between first and second electrodes, impedance measurement module 92 may measure a resulting voltage, and processor 80 may calculate a resistance based upon the current amplitude of the pulse and the measured amplitude of the resulting voltage. Impedance measurement module 92 may include circuitry for measuring amplitudes of resulting currents or voltages, such as sample and hold circuitry.

In these examples, signal generator 84 delivers signals that do not necessarily deliver stimulation therapy to heart 12, due to, for example, the amplitudes of such signals and/or the timing of delivery of such signals. For example, these signals may comprise sub-threshold amplitude signals that may not stimulate heart 12. In some cases, these signals may be delivered during a refractory period, in which case they also may not stimulate heart 12. IMD 16 may use defined or predetermined pulse amplitudes, widths, frequencies, or electrode polarities for the pulses delivered for these various impedance measurements. In some examples, the amplitudes and/or widths of the pulses may be sub-threshold, e.g., below a threshold necessary to capture or otherwise activate tissue, such as cardiac tissue of heart 12.

In certain cases, IMD 16 may collect impedance values that include both a resistive and a reactive (i.e., phase) component. In such cases, IMD 16 may measure impedance during delivery of a sinusoidal or other time varying signal by signal generator 84, for example. Thus, as used herein, the term “impedance” is used in a broad sense to indicate any collected, measured, and/or calculated value that may include one or both of resistive and reactive components.

Processor 80 may control signal generator 84 to deliver the test pulses for impedance measurement according to the integrity test parameters 83 stored in memory 82. For example, processor 80 may control the timing or amplitude of test pulses based on the integrity test parameters 83. The integrity test parameters 83 may, in some examples, specify a period of time, e.g., a window, subsequent a detected event, which may be an R-wave or noise, in which one or more test pulses may be delivered. The duration of the period may be selected as appropriate to determine the most accurate impedance values. Furthermore, by controlling the timing of test pulses in this manner, interference with the accuracy of impedance measurements by intrinsic cardiac signals may be avoided.

Processor 80 may compare the impedances measured from each of the test pulses to an impedance threshold, and evaluate the integrity of the sensing configuration, or more generally lead integrity, based upon the comparison. The impedance threshold may be a predetermined, e.g., user-programmed, value, or a value determined based on previous impedance measurements, such as periodic impedance measurements. In some examples, the measured impedance that is compared to the threshold is an average or median of a number of measured impedances. In some examples, processor 80 determines trends of impedance measurements, or statistical or other processed values determined based on impedance measurements, to determine whether a lead related condition is present.

In one example, processor 80 may detect a lead related condition if at least two of the following criteria are met within the past 60 days: a lead impedance measurement is less than approximately 50% or greater than approximately 175% of a baseline impedance value, a sensing integrity counter is incremented by at least approximately 30 within a period of three consecutive days or less, or sensing module 86 detects at least two non-sustained high-rate cardiac episodes with a 4-beat average cardiac interval of less than approximately 220 milliseconds. However, any lead integrity criteria, utilizing any parameters indicative of the integrity of the lead, may be utilized to detect a lead related condition.

In some examples, a lead related condition may be detected based on a single measurement, e.g., based on a single impedance measurement outside of a range stored in memory 82. In other examples, a lead related condition may be detected based a threshold number of lead integrity episodes or a threshold number of lead integrity episodes within a predetermined period of time. In some examples, processor 80 may detect a lead related condition if a single integrity criterion has been met. In other examples, processor 80 may require multiple integrity criteria to be met in conjunction, e.g., within a common time period, to detect a lead related condition.

In some examples, processor 80 may detect varying degrees of lead related conditions based on different sets of criteria. For example, processor 80 may detect a lead related condition indicative of a possible lead integrity issue based on a first set of criteria and increase a frequency of cardiac capture in response to detection. Processor 80 may also detect a lead related condition indicative of a more probable lead integrity issue based on a second set of criteria, e.g., a more rigid set of criteria, and trigger a lead integrity alert based on the detection. In other examples, processor 80 may trigger a lead integrity alert if a threshold number of lead related conditions are detected or a threshold number of lead related conditions are detected within a period of time. By increasing a frequency of capture detection prior to a full lead integrity alert, processor 80 may determine whether LOC or intermittent LOC precedes a lead integrity alert. This may lead to a better understanding of when capture is lost due to lead problems.

Example methods of detecting lead related conditions are described in U.S. Patent Application No. 20090299432 by Stadler et al., entitled, “IMPEDANCE VARIABILITY ANALYSIS TO IDENTIFY LEAD-RELATED CONDITIONS,” which was published on Dec. 3, 2009; U.S. Patent Application No. 20090299422 by Ousdigian et al., entitled, “ELECTROGRAM STORAGE FOR SUSPECTED NON-PHYSIOLOGICAL EPISODES,” which published on Dec. 3, 2009; U.S. Patent Application No. 20080161870 by Gunderson, entitled, “METHOD AND APPARATUS FOR IDENTIFYING CARDIAC AND NON-CARDIAC OVERSENSING USING INTRACARDIAC ELECTROGRAMS,” which published on Jul. 3, 2008; U.S. Patent Application No. 20060116733 by Gunderson, entitled METHOD AND APPARATUS FOR IDENTIFYI8NG LEAD-RELATED CONDITIONS USING PREDICTION AND DETECTION CRITERIA, which published on Jun. 1, 2006; and U.S. Patent Application No. 20050137636 by Gunderson et al., entitled METHOD AND APPARATUS FOR IDENTIFYING LEAD-RELATED CONDITIONS USING IMPEDANCE TREANDS AND OVERSENSING CRITERIA, which published on Jul. 23, 2005. Each of these applications is incorporated herein by reference in its entirety.

Processor 80 may periodically or continuously monitor lead integrity. In some examples, if processor 80 detects a lead related condition, processor 80 may trigger a lead integrity alert. A lead integrity alert may provide advance warning of a potential lead fracture or other lead related condition. A lead integrity alert may be an alert presented to a user of a computing device that communicates with IMD 16, e.g., programmer 24.

Referring again to FIG. 4, telemetry module 88 in IMD 16 includes any suitable hardware, firmware, software or any combination thereof for communicating with another device, such as programmer 24 (FIG. 1). Under the control of processor 80, telemetry module 88 may receive downlink telemetry from and send uplink telemetry to programmer 24 with the aid of an antenna, which may be internal and/or external. Processor 80 may provide the data to be uplinked to programmer 24 and the control signals for the telemetry circuit within telemetry module 88, e.g., via an address/data bus. In some examples, telemetry module 88 may provide received data to processor 80 via a multiplexer.

Processor 80 may transmit atrial and ventricular heart signals (e.g., electrocardiogram signals) produced by atrial and ventricular sense amp circuits within sensing module 86 to programmer 24. Programmer 24 may interrogate IMD 16 to receive the heart signals. Processor 80 may store heart signals within memory 82, and retrieve stored heart signals from memory 82. Processor 80 may also generate and store marker codes indicative of different cardiac events that sensing module 86 detects, and transmit the marker codes to programmer 24. An example pacemaker with marker-channel capability is described in U.S. Pat. No. 4,374,382 to Markowitz, entitled, “MARKER CHANNEL TELEMETRY SYSTEM FOR A MEDICAL DEVICE,” which issued on Feb. 15, 1983 and is incorporated herein by reference in its entirety.

In addition, processor 80 may transmit integrity testing information to programmer 24 via telemetry module 88. In some examples, telemetry module 88 may transmit an alert to programmer 24 indicating an integrity issue with an electrode configuration, or programmer 24 may provide such an alert in response to the testing information received from IMD 16. This alert may prompt the user to reprogram IMD 16 to use a different sensing or therapy configuration, or perform some other function to address the possible integrity issue. In some examples, IMD 16 may signal programmer 24 to further communicate with and pass the alert through a network such as those available under the trade designation Medtronic CareLink Network from Medtronic, Inc., of Minneapolis, Minn., or some other network linking patient 14 to a clinician. In some examples, telemetry module 88 may transmit an alert to programmer 24 when a pacing capture threshold increased and/or loss of capture has been detected, e.g., in response to a lead related condition. Telemetry module 88 may also transmit other information regarding cardiac capture, such as times that loss of capture occurred, frequency of loss of capture, and pacing threshold values for alternate pacing electrode configurations.

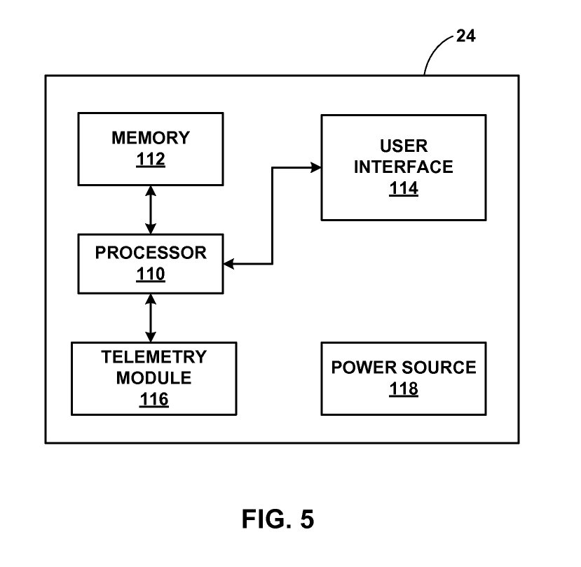

FIG. 5 is functional block diagram illustrating an example configuration of programmer 24. As shown in FIG. 5, programmer 24 may include a processor 110, a memory 112, a user interface 114, a telemetry module 116, and a power source 118. Programmer 24 may be a dedicated hardware device with dedicated software for programming IMD 16. Alternatively, programmer 24 may be a commercially available off-the-shelf computing device running an application that enables programmer 24 to program IMD 16.

A user may use programmer 24 to select therapy programs (e.g., sets of stimulation parameters), generate new therapy programs, modify therapy programs through individual or global adjustments or transmit the new programs to a medical device, such as IMD 16 (FIG. 1). The clinician may interact with programmer 24 via user interface 114, which may include display to present graphical user interface to a user, and a keypad or another mechanism for receiving input from a user.

The user may also use programmer 24 to adjust or control the integrity testing performed by IMD 16. For example, the user may use programmer 24 to program the number of test pulses, the timing of test pulses, the parameters of each test pulse, or any other aspects of the impedance measurements of lead integrity tests. In this manner, the user may be able to finely tune the integrity test to the specific condition of patient 14.

In addition, the user may receive an alert from IMD 16 indicating a potential integrity issue with the current sensing configuration via programmer 24. The user may respond to IMD 16 by selecting an alternative sensing configuration via programmer 24 or overriding the integrity issue if a cardiac event is occurring. Alternatively, IMD 16 may automatically select an alternative sensing configuration. Programmer 24 may prompt the user to confirm the selection of the alternative sensing configuration.

Programmer 24 may also receive information regarding capture detection, e.g., subsequent to a detected lead related condition. Such information may include, e.g., times that loss of capture occurred, frequency of loss of capture, and pacing threshold values for alternate pacing electrode configurations. In some examples, programmer 24 may receive indications from IMD 16 if a pacing capture threshold increased and/or loss of capture occurred subsequent to a lead related condition.

Processor 110 can take the form one or more microprocessors, DSPs, ASICs, FPGAs, programmable logic circuitry, or the like, and the functions attributed to processor 110 herein may be embodied as hardware, firmware, software or any combination thereof. Memory 112 may store instructions that cause processor 110 to provide the functionality ascribed to programmer 24 herein, and information used by processor 110 to provide the functionality ascribed to programmer 24 herein. Memory 112 may include any fixed or removable magnetic, optical, or electrical media, such as RAM, ROM, CD-ROM, hard or floppy magnetic disks, EEPROM, or the like. Memory 112 may also include a removable memory portion that may be used to provide memory updates or increases in memory capacities. A removable memory may also allow patient data to be easily transferred to another computing device, or to be removed before programmer 24 is used to program therapy for another patient.

Programmer 24 may communicate wirelessly with IMD 16, such as using RF communication or proximal inductive interaction. This wireless communication is possible through telemetry module 116, which may be coupled to an internal antenna or an external antenna. An external antenna that is coupled to programmer 24 may correspond to the programming head that may be placed over heart 12, as described above with reference to FIG. 1. Telemetry module 116 may be similar to telemetry module 88 of IMD 16 (FIG. 4).

Telemetry module 116 may also communicate with another computing device via wireless communication techniques, or direct communication through a wired connection. Examples of local wireless communication techniques that may be employed to facilitate communication between programmer 24 and another computing device include RF communication according to the 802.11 or Bluetooth specification sets, infrared communication, e.g., according to the IrDA standard, or other standard or proprietary telemetry protocols. In this manner, other external devices may be capable of communicating with programmer 24 without needing to establish a secure wireless connection. An additional computing device in communication with programmer 24 may be a networked device such as a server capable of processing information retrieved from IMD 16.

In some examples, processor 110 of programmer 24 and/or one or more processors of one or more networked computers may perform all or a portion of the techniques described herein with respect to processor 80 and IMD 16. For example, processor 110 or another processor may receive voltages or currents measured by IMD 16 to calculate impedance measurements, or may receive impedance measurements from IMD 16. Processor 110 or another processor may compare impedance measurements to evaluate lead integrity using any of the techniques described herein. Processor 110 or another processor may also receive indications of lead related conditions, control IMD 16 to increase a frequency of capture detection subsequent to a detected lead related condition, receive capture information, control IMD 16 to switch sensing or therapy configurations, or may provide an alert, based on the detected lead integrity and/or capture management information, according to any of the techniques described herein.

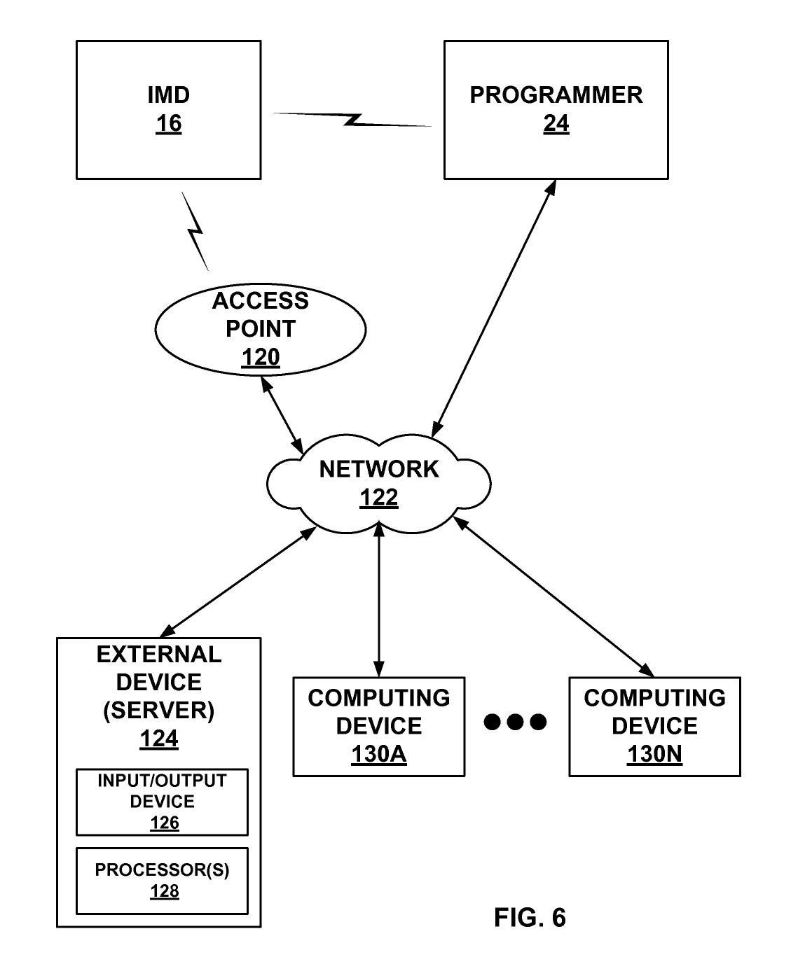

FIG. 6 is a block diagram illustrating an example system that includes an external device, such as a server 124, and one or more computing devices 130A-130N, that are coupled to IMD 16 and programmer 24 shown in FIG. 1 via a network 122. In this example, IMD 16 may use its telemetry module 88 to communicate with programmer 24 via a first wireless connection, and to communicate with an access point 120 via a second wireless connection. In the example of FIG. 6, access point 120, programmer 24, server 124, and computing devices 130A-130N are interconnected, and able to communicate with each other, through network 122. In some cases, one or more of access point 120, programmer 24, server 124, and computing devices 130A-130N may be coupled to network 122 through one or more wireless connections. IMD 16, programmer 24, server 124, and computing devices 130A-130N may each include one or more processors, such as one or more microprocessors, DSPs, ASICs, FPGAs, programmable logic circuitry, or the like, that may perform various functions and operations, such as those described herein.

Access point 120 may include a device that connects to network 122 via any of a variety of connections, such as telephone dial-up, digital subscriber line (DSL), or cable modem connections. In other embodiments, access point 120 may be coupled to network 122 through different forms of connections, including wired or wireless connections. In some embodiments, access point 120 may be co-located with patient 14 and may include one or more programming units and/or computing devices (e.g., one or more monitoring units) that may perform various functions and operations described herein. For example, access point 120 may include a home-monitoring unit that is co-located with patient 14 and that may monitor the activity of IMD 16.

In some examples, server 124 or computing devices 130 may perform any of the various functions or operations described herein. As shown in FIG. 6, server 124 may include an input/output device 126 and processor(s) 128, similar to those in programmer 24. A user may interact with server 124 via input/output device 126, similar to programmer 24. In addition, processors 128 may perform any calculations, data processing, communication relay, or any other task required to treat or monitor patient 14.

For example, server 124 or computing devices 130, processor 110 or another processor may receive, from IMD 16, voltages or currents measured by IMD 16 to calculate impedance measurements, or may receive impedance measurements from IMD 16 via network 122. Server 124 or computing devices 130 may compare impedance measurements to evaluate lead integrity using any of the techniques described herein. Server 124 or computing devices 130 may also receive indications of lead related conditions, control IMD 16 to increase a frequency of capture detection subsequent to a detected lead related condition, receive capture information, control IMD 16 to switch sensing or therapy configurations, or may provide an alert, based on the detected lead integrity and/or capture management information, according to any of the techniques described herein. In some examples, server 124 may provide some or all of this functionality, and provide alerts to interested users, e.g., a physician for patient 14 or technician for a manufacturer of IMD 16 or leads 18, 20 and 22, via network 122 and computing devices 130.

In some cases, server 124 may provide a secure storage site for archival of lead integrity information, such as impedance measurements, and capture management information, that has been collected from IMD 16 and/or programmer 24. Network 122 may include a local area network, a wide area network, or a global network, such as the Internet. In some cases, programmer 24 or server 124 may assemble lead integrity and/or capture management information in web pages or other documents for viewing by and trained professionals, such as clinicians, via viewing terminals associated with computing devices 130A-130N. The system of FIG. 6 may be implemented, in some aspects, with general network technology and functionality similar to that provided by the network available under the trade designation Medtronic CareLink Network from Medtronic, Inc., of Minneapolis, Minn.

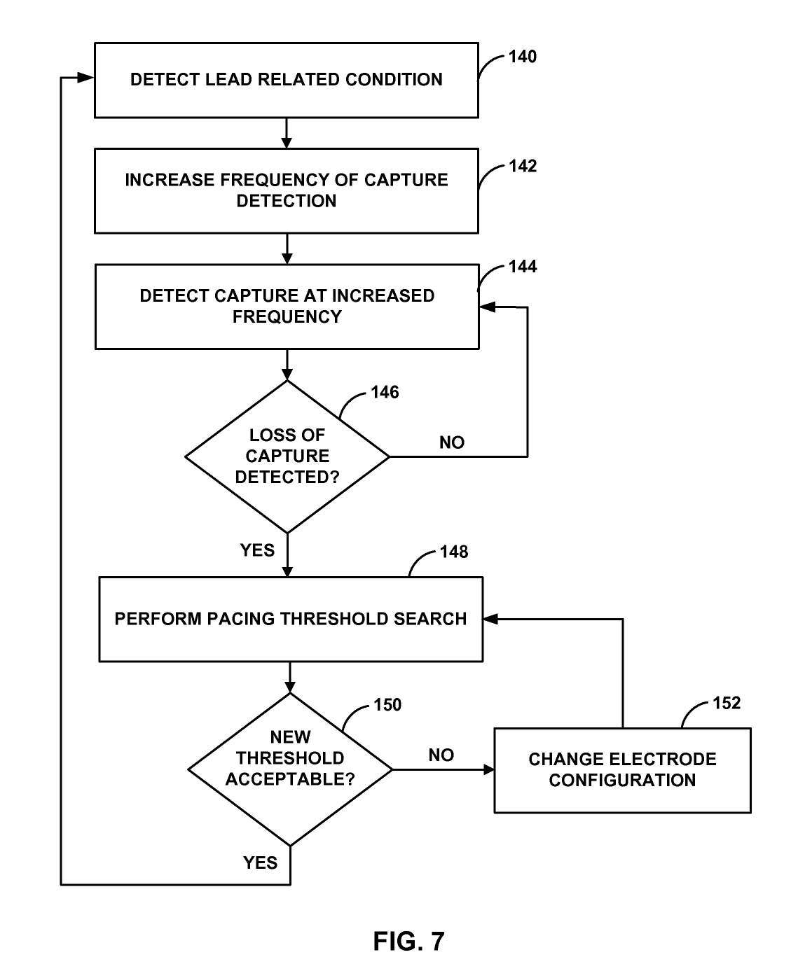

FIG. 7 is a flow diagram of an example method of increasing a frequency of capture detection in response to a lead related condition. The example method of FIG. 7 is described as being performed by processor 80 and sensing module 86 of IMD 16. In other examples, one or more other processors of one or more other devices may implement all or part of this method.

Processor 80 detects a lead related condition (140). The lead related condition may be based on a single measurement or a series of measurements. For example, processor 80 may detect a lead related condition based on a single impedance measurement outside of the bounds stored in memory 82. As another example, processor 80 may detect a lead related condition based on the frequency of non-sustained high-rate cardiac episodes or the frequency of short ventricular intervals counted on a sensing integrity counter. In some examples, processor 80 may detect a lead related condition if a single integrity criterion has been met. In other examples, processor 80 may require multiple integrity criteria to be met to detect a lead related condition.

In response to the detected lead related condition, processor 80 may increase a frequency of capture detection (144). For example, processor 80 and one or more capture measurement channels 87 may detect cardiac capture on a substantially continuous basis, e.g., on approximately an every heartbeat basis, subsequent to a detected lead related condition. As an alternative, processor 80 and one or more capture measurement channels 87 may detect capture on a periodic basis with an increased frequency, e.g., approximately every fifteen minutes, approximately every one minute, subsequent to a detected lead related condition. Since increased pacing capture thresholds and/or loss of capture may occur subsequent to lead related conditions, increasing a frequency of capture detection may allow processor 80 to detect and respond to these issues.

Processor 80 and one or more capture measurement channels of sensing module 86 may detect capture at the increased frequency (144). If loss of capture is detected (146), processor 80 may perform a pacing threshold search (148). Performing a pacing threshold search may allow processor 80 to determine whether increasing the magnitude of the pacing pulse may effectively maintain capture of heart 12. If a new capture threshold is determined (150), processor 80 may return to detecting capture at its normal frequency, e.g., until lead related condition is detected (140).

If the pacing threshold search does not obtain an acceptable capture threshold for the selected electrode configuration, e.g., capture can not be obtained at the maximum magnitude or capture can only be obtained at a high magnitude, processor 80 may modify the electrode configuration used for delivering pacing therapy (152) and perform a pacing threshold search on the new electrode configuration (148). Processor 80 may select the new electrode configuration based on information associated with the lead related condition.

As one example, if the lead related condition indicates that there may be an integrity issue associated with the conductor associated with RV ring electrode 40 when tip electrode 42 and ring electrode 40 are being used to deliver pacing signals, processor 80 may modify the electrode configuration to utilize the RV coil electrode 62 instead and pace using tip electrode 42 and coil electrode 62, e.g., in an integrated bipolar system. As another example in this scenario, processor 80 may modify the electrode configuration to utilize housing electrode 58 instead of RV ring electrode 40 and pace using tip electrode 42 and housing electrode 58, e.g., in an unipolar system. If subsequent threshold measurements were acceptable, processor 80 may return the capture detection frequency to a normal frequency.

As another example, the lead related condition may indicate that there is an integrity issue associated with the conductor associated with RV tip electrode 42 when tip electrode 42 and ring electrode 40 are being used to deliver pacing signals. In this scenario, it may be less likely that an acceptable capture threshold may be found using RV ring electrode 40 or RV coil electrode 62 as the pacing cathode. Therefore, processor 80 may switch RV ring electrode 42 from the pacing configuration and test RV tip electrode 42 in combination with RV coil electrode 62 and/or RV tip electrode 42 in combination with housing electrode 58 to determine whether an acceptable exists for either electrode configuration. However, if RV ring electrode 40 and/or RV coil electrode 62 are in contact with tissue, it is possible that that an acceptable capture threshold may be found using RV ring electrode 40 or RV coil electrode 62 as the pacing cathode. Therefore, if an acceptable threshold could not be determined from RV tip electrode 42 in combination with RV coil electrode 62 or RV tip electrode 42 in combination with housing electrode 58, processor 80 may test the combination of RV ring electrode 40 and RV coil electrode 62 and/or the combination of RV ring electrode 40 and housing electrode 58 to determine whether acceptable pacing thresholds exist for these electrode configurations.

The method described with respect to FIG. 7 is merely one example. In an alternative example, processor 80 may automatically change the electrode configuration used for delivering pacing therapy in response to detecting the lead related condition. Processor 80 may monitor capture at the increased frequency on the new electrode configuration, e.g., until processor 80 determines that capture is consistently obtained. In any example, processor 80 may return to detecting capture at its normal frequency, or otherwise decrease the frequency of capture detection, in response to determining that substantially continuous capture is achieved, e.g., after a predetermined period of time without detection of loss of capture.

In some examples in which capture detection is performed on a less than beat-to-beat basis, processor 80 may increase a frequency of capture detection by increasing a frequency of pacing threshold search. Processor 80 may determine whether loss of capture occurred based on the pacing threshold value.

Various examples have been described. These and other examples are within the scope of the following claims.

1. A method comprising:
delivering a pacing therapy to a heart of a patient;
periodically determining whether the pacing therapy captures the heart of the patient;
detecting a lead related condition; and
in response to the detection of the lead related condition, increasing a frequency of determining whether the pacing therapy captures the heart.

2. The method of claim 1, wherein increasing the frequency comprises increasing from periodic detection at a first frequency to periodic detection at a second frequency.

3. The method of claim 2, wherein the periodic capture detection at the second frequency comprises capture detection on approximately an every fifteen minute basis.

4. The method of claim 2, wherein the periodic capture detection at the second frequency comprises capture detection on approximately an every minute basis.

5. The method of claim 1, wherein increasing the frequency comprises increasing from periodic detection to substantially continuous detection.

6. The method of claim 5, wherein substantially continuous detection comprises detection at approximately every heart beat.

7. The method of claim 1, wherein detecting the lead related condition comprises detecting at least one of an impedance measurement outside of a normal range, an open circuit, a short circuit, a non-sustained high-rate cardiac episode, or a short cardiac interval.

8. The method of claim 1, wherein detecting the lead related condition comprises at least one of detecting a threshold number of lead integrity episodes or detecting a threshold number of lead integrity episodes within a predetermined period of time, wherein the lead integrity episodes comprise at least one of non-sustained high-rate cardiac episodes or short cardiac intervals.

9. The method of claim 1, further comprising triggering a lead integrity alert in response to the lead related condition.

10. The method of claim 1, further comprising changing an electrode configuration for delivering the pacing therapy in response to detecting the lead related condition.

11. A system comprising:
a stimulation generator configured to deliver a pacing therapy to a heart of a patient;
an electrical stimulation lead coupled to the stimulation generator, wherein the stimulation generator delivers the pacing therapy via the electrical stimulation lead;
a sensing module configured to sense an indicator of integrity of the lead; and
a processor configured to periodically determine whether the pacing therapy captures the heart of the patient, identify a lead related condition based on the sensed indicator, and increase a frequency of determining whether the pacing therapy captures the heart in response to the detection of the lead related condition.

12. The system of claim 11, further comprising an implantable medical device that comprises the signal generator.

13. The system of claim 11, wherein the implantable medical device comprises the sensing module and the processor and is coupled to the electrical stimulation lead.

14. The system of claim 11, wherein the implantable medical device comprises at least one of a pacemaker, cardioverter, or defibrillator.

15. The system of claim 11, wherein the processor increases the frequency from periodic detection at a first frequency to periodic detection at a second frequency.

16. The system of claim 15, wherein the periodic capture detection at the second frequency comprises capture detection on approximately an every fifteen minute basis.

17. The system of claim 15, wherein the periodic capture detection at the second frequency comprises capture detection on approximately an every minute basis.

18. The system of claim 11, wherein the processor increases the frequency from periodic detection to substantially continuous detection.

19. The system of claim 18, wherein substantially continuous detection comprises detection at approximately every heart beat.

20. The system of claim 11, wherein the lead related condition comprises at least one of an impedance measurement outside of a normal range, a detected open circuit, a detected short circuit, a non-sustained high-rate cardiac episode, or a short interval.

21. The system of claim 11, wherein the lead related condition comprises at least one of a threshold number of lead integrity episodes or a threshold number of lead integrity episodes within a predetermined period of time, wherein the lead integrity episodes comprise at least one of non-sustained high-rate cardiac episodes or short cardiac intervals.

22. The system of claim 11, further comprising a programmer, the programmer including a user interface, wherein the user interface is configured to provide an alert in response to the lead related condition.

23. The system of claim 11, wherein the processor modifies an electrode configuration for delivering the pacing therapy in response to the detected lead related condition and controls the stimulation generator to deliver the pacing therapy using the modified electrode configuration.

24. A system comprising:
means for delivering a pacing therapy to a heart of a patient;
means for periodically determining whether the pacing therapy captures the heart of the patient;
means for detecting a lead related condition; and
means for increasing a frequency of determining whether the pacing therapy captures the heart in response to the detection of the lead related condition.

25. A computer-readable storage medium comprising instructions that, when executed, cause a programmable processor to:
deliver a pacing therapy to a heart of a patient;
periodically determine whether the pacing therapy captures the heart of the patient;
detect a lead related condition; and
in response to the detection of the lead related condition, increase a frequency of determining whether the pacing therapy captures the heart.
